(19) United States
(12) Patent Application Publication
(10) Pub. No.: US 2007/0048357 A1
(43) Pub. Date: Mar. 1, 2007

(54) Fibrous wiping products

(75) Inventor: Thomas G. Shannon, Neenah, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/216,530

(22) Filed: Aug. 31, 2005

(51) Int. Cl. A61K 9/70; A61F 13/00

(52) U.S. Cl. 424/443

(57) ABSTRACT

Sheet-like products are disclosed having a controlled equilibrium moisture content. The sheet-like product may comprise a dry product or a pre-moistened product. The sheet-like product, for instance, may comprise a facial tissue, a bath tissue, a paper towel, a napkin, or a wet wipe, such as pre-moistened bath tissue. In order to control the equilibrium moisture content of the product, a mixture of salts containing at least one deliquescent salt or a single deliquescent salt, namely lithium bromide, are incorporated into the product. By mixing different salts together, the mixture may have a relatively low deliquescence point allowing the salts to control the equilibrium moisture content of the sheet-like product even in relatively low humidity environments.

### BACKGROUND OF THE INVENTION

Wet wipes and moist toilet paper suffer from dry-out when exposed to air for extended periods of time. This is particularly noticeable with moist toilet paper, for example, because the leading edge of the sheet that remains outside the dispenser is prone to drying out between uses, which leaves a negative consumer impression. While considerable effort is given to designing dispensers that eliminate or at least minimize the exposure of the leading edge of the sheet to ambient conditions, some exposure is inevitable for some products like moist toilet paper because complete enclosure of the product is undesirable from the standpoint of the user.

With regard to dry wiping products, such as facial tissue, dry toilet paper, table napkins, paper towels and the like, moisture in the sheet is known to impart a softness benefit by plasticizing the fibers. While it is known to add humectants to tissue products to improve the hand feel, humectants do not absorb appreciable quantities of water relative to their weight. Hence, very large amounts of the humectant material are required to absorb moisture in amounts sufficient to be effective. In addition, humectant materials do not form solutions with the water but rather exist as water/humectant complexes. Hence the water is bound to the humectant material and does not impart the same effect as free water in the sheet. Further, if the humectant material is a solid particulate, it will remain as a solid particulate in the sheet and can impart a gritty feel to the sheet.

Therefore, there is a need for an economically feasible dry wiping product having a high equilibrium moisture content so as to exhibit improved softness and pliability without increasing the grittiness of the product. Furthermore, with regard to moist wiping products, there is a need for a sheet that has a sufficiently high equilibrium moisture content such that the sheet maintains a moist feel when exposed to ambient conditions for an extended period of time.

### SUMMARY OF THE INVENTION

In general, the present disclosure is directed to wiping products and other sheet-like materials that are capable of retaining moisture. For example, in one embodiment, a fibrous product comprises a base web that contains cellulosic fibers. In order to improve the fibrous properties of the base web, a mixture of deliquescent salts is placed in association with the base web. The deliquescent salts are present in an amount sufficient to increase the equilibrium moisture content of the base web. As used herein, the “equilibrium moisture content” represents the moisture content of the base web at a particular relative humidity and at a particular temperature. At equilibrium, the amount of moisture within the sheet will not change with time at the same humidity condition. The equilibrium moisture content is expressed as a weight percent of the dry sheet including the mixture of deliquescent salts and any additional non-volatile components.

Increasing the moisture content of the base web imparts a noticeable wet feel to the product. Increasing the moisture content produces a product that exhibits improved softness and pliability.

In accordance with the present disclosure, a deliquescent salt, a mixture of deliquescent salts or a mixture of deliquescent and non-deliquescent salts is incorporated into the base web such that the salt or salt mixture has a deliquescence point of less than about 30%. As used herein, the “deliquescence point” is the relative humidity below which the salt will effloresce and lose water to become a solid material. By using a mixture of deliquescent salts, the mixture may have a deliquescence point lower than the deliquescence point of the individual salts. In some cases, non-deliquescent salts may be blended with a deliquescent salt or salts to form a mixture having a deliquescence point below the deliquescent salt or mixture of deliquescent salts. By maintaining a relatively low deliquescence point, the mixture of salts does not effloresce at low relative humidity allowing the sheet to maintain its superior softness due to the high equilibrium moisture content and lack of any solid salt that might impart a gritty feel to the product. In particular embodiments, for instance, the deliquescence point of the mixture of deliquescent salts may be less than about 20% relative humidity, less than about 15% relative humidity, and, in one embodiment, less than about 10% relative humidity.

The mixture of deliquescent salts may comprise, for instance, at least two salts selected from the group consisting of calcium chloride, magnesium chloride, potassium acetate, lithium bromide, lithium chloride, potassium carbonate, magnesium nitrate, sodium bromide, potassium iodide, sodium chloride, potassium chloride, potassium sulfate, an aluminate, sodium acetate, and ammonium acetate. In one embodiment, the mixture of deliquescent salts at least contains calcium chloride or alternatively may at least contain magnesium chloride.

The base web that is placed in association with the mixture of deliquescent salts may vary depending upon the particular application. For example, in one embodiment, the base web may comprise a dry tissue product, such as a facial tissue or a bath tissue. The tissue product may have, for instance, a dry sheet bulk of at least about 2 cm³/g and may consist essentially of pulp fibers. When combined with the mixture of deliquescent salts, the tissue web may have an equilibrium moisture content of from about 10% to about 30% at a relative humidity between 20% and 80% at 72° F. The base web may also have a moisture content of from about 10% to about 30% at a relative humidity between 5% and 30% at 72° F. When determining the equilibrium moisture content of a dry product, the dry sample may be conditioned at least four hours at the TAPPI standard conditions prior to determining the equilibrium moisture content of the base web.

In an alternative embodiment, the product may comprise a wet wipe and, when combined with the mixture of deliquescent salts, the base web may have an equilibrium moisture content of from about 30% to about 200% at a relative humidity between 5% and 30% at 720F. For wet products, the wet sample sheets may first be dried at 100° C. for a minimum of one hour. The dry sample can then be conditioned at least four hours at controlled temperature and humidity conditions prior to determining the equilibrium moisture content of the sheet.

The amount of deliquescent salts present in the base web can vary depending upon the particular application and various factors. In general, for instance, the mixture of deliquescent salts may be present in the base web in an amount from about 2% to about 100% by weight. In one embodiment, in addition to containing the deliquescent salts, the base web may also contain a friction reducing agent.

Other features and aspects of the present invention are discussed in greater detail below.

### DETAILED DESCRIPTION

It is to be understood by one of ordinary skill in the art that the present discussion is a description of exemplary embodiments only, and is not intended as limiting the broader aspects of the present invention.

The present disclosure is generally directed to sheet-like products that have enhanced moisture retention properties under conditions of low relative humidity. The sheet-like product in one embodiment may comprise a tissue product. The tissue product, for instance, may be a facial tissue or a bath tissue. The sheet-like product may also comprise a pre-moistened wipe.

In accordance with one embodiment of the present disclosure, in order to increase the moisture retention properties of the product at low relative humidity, a deliquescent salt, a mixture of deliquescent and non-deliquescent salts, or a mixture of deliquescent salts are incorporated into the product. By combining at least two different salts together, the present inventor has found that the deliquescence point of the mixture may be lower than the deliquescence point of the individual components. Thus, two or more salts may be combined in order to control and lower the deliquescence point. Lowering the deliquescence point ensures that the deliquescent salts will not effloresce and solidify and cause the product to feel somewhat gritty even when exposed to lower relative humidity.

In other embodiments, a single deliquescent salt may be incorporated into the product that has by itself a very low deliquescence point. For instance, in one embodiment, the deliquescent salt may comprise a lithium salt, such as lithium bromide.

As used herein, a “deliquescent salt” refers to a salt capable of absorbing moisture from the air to form a solution at 72° F. and at 50% relative humidity.

Should a sheet-like product containing a deliquescent salt be exposed to an environment in which the relative humidity is below the deliquescence point, the salt will effloresce and become a solid material. For example, residential heating in the winter and dry desert areas may have a relative humidity below 20%. The deliquescence point of many deliquescent salts, on the other hand, is greater than 20%. For instance, one of the more common deliquescent salts is calcium chloride, which has a deliquescence point of approximately 30% relative humidity at 72° F. Thus, a sheet-like product containing just calcium chloride may not have an increased equilibrium moisture level when the relative humidity of the environment drops below about 30% relative humidity. Combining calcium chloride with other deliquescent salts or non-deliquescent salts, however, creates a mixture having a deliquescence point lower than the deliquescence point of calcium chloride alone.

In various embodiments, for instance, a mixture of salts including at least one deliquescent salt may be prepared and incorporated into a sheet-like product wherein the mixture has a deliquescence point of less than about 30% relative humidity, such as less than about 25% relative humidity, such as less than about 20% relative humidity. In still other embodiments, a mixture of salts may be prepared that has a deliquescence point of less than about 15% relative humidity, such as less than about 12% relative humidity.

Examples of deliquescent and non-deliquescent salts that may be combined together and incorporated into a sheet-like product include various chloride salts, acetate salts, carbonate salts, nitrate salts, bromide salts, iodide salts, and the like. The salt may be formed using any suitable alkali metal, alkaline earth metal, or ammonium.

Particular examples of deliquescent salts that may be used in the present invention include calcium chloride, magnesium chloride, potassium acetate, lithium bromide, lithium chloride, magnesium nitrate, an aluminate, and ammonium acetate. Examples of non-deliquescent salts that may be combined with the deliquescent salt include potassium carbonate, sodium bromide, potassium iodide, sodium chloride, potassium chloride, potassium sulfate, sodium acetate, and the like. In one embodiment, only two salts may be incorporated into the sheet-like product. In other embodiments, however, the sheet-like product may contain three or more of the salts. When blending salts, it is essential that no interactions occur between the salts that create formation of insoluble precipitates, such formation causing softness deficiency in the product and often destroying the deliquescent character of the salt blend.

For exemplary purposes only, in one embodiment, magnesium chloride may be combined with calcium chloride to form a mixture of deliquescent salts in accordance with the present disclosure. When incorporated into a sheet-like product, the weight ratio of magnesium chloride to calcium chloride may be from about 1:1 to about 30:1, such as from about 4:1 to about 25:1. Magnesium chloride has a deliquescence point of about 33% relative humidity, while calcium chloride has a deliquescence point of about 30% relative humidity. When combined together in the above ratios, however, the mixture may have a deliquescence point of less than about 20% relative humidity, such as less than about 12% relative humidity. Thus, combining various salts together can have a very surprising effect on the deliquescence point.

In an alternative embodiment, the deliquescent salt magnesium chloride may be combined with the non-deliquescent salt sodium chloride. In this embodiment, the weight ratio of magnesium chloride to sodium chloride may be from about 1:1 to about 30:1, such as from about 2:1 to about 25:1. Sodium chloride has a deliquescence point of about 75% relative humidity. When combined with magnesium chloride, however, the mixture can have a deliquescence point of less than 30% relative humidity, such as less than about 20% relative humidity.

In other embodiments, calcium chloride may be combined with sodium chloride or potassium acetate may be combined with magnesium chloride, sodium chloride, or calcium chloride. The above mixtures of salts may all have weight ratios of from about 1:1 to about 25:1, such as from about 3:1 to about 12:1. Further, it should be understood that in addition to only combining two salts, various advantages and benefits may be achieved by combining three or more salts together.

In addition to combining a mixture of salts containing at least one deliquescent salt into a sheet-like product, such as a tissue product, in still another alternative embodiment of the present invention, a single deliquescent salt, namely lithium bromide, is incorporated into the sheet-like product. Lithium bromide has been found to have a deliquescence point of less than 7% relative humidity. Thus, lithium bromide may be incorporated alone into a sheet-like product with little need to combine with other deliquescent salts in view of its very low deliquescence point.

When the salts are incorporated into a sheet-like product, such as a dry tissue product or a pre-moistened wipe, the salts can dramatically increase the equilibrium moisture content of the product providing the product with a softer feel.

For example, dry products treated in accordance with the present invention may have an equilibrium moisture content of from about 10% to about 30% at relative humidities between about 20% to about 80% at 72° F. For dry tissue products, equilibrium moisture contents of between 10% to about 30% are found to give the optimal perception of softness to the product. At equilibrium moisture levels below 10% only minimal improvements in softness are noticeable while at equilibrium moisture levels above about 30% the products take on a noticeable damp feel that detracts from a favorable perception for use as a dry tissue product. When the deliquescent salts alone or together or mixed with non-deliquescent salts have a relatively low deliquescence point, the dry products can also have an equilibrium moisture content of from about 10% to about 30% at relative humidities between about 5% and about 30% at 72° F.

For pre-moistened products, on the other hand, after being treated with deliquescent salts, the product can have an equilibrium moisture content of from about 30% to about 200% at relative humidities between 7% and 80% at 72° F., and particularly at relative humidities between about 7% and 30% at 72° F.

The amount of salts incorporated into a sheet-like product in accordance with the present disclosure may vary depending upon various factors, including the desired equilibrium moisture content. For example, the salts containing at least one deliquescent salt may be incorporated into the sheet-like product in an amount from about 2% to about 150% by weight of dry fiber or greater, such as from about 2% to about 125 dry weight percent, such as from about 5% to about 100 dry weight percent. In some applications, for instance, the deliquescent salts may be added to the sheet-like product in an amount from about 10 dry weight percent to about 50 dry weight percent. The specific amount of salts incorporated into the product is not overly critical so long as the desired equilibrium moisture content is achieved at the desired deliquescence point.

The salts containing at least one deliquescent salt may be incorporated into the sheet-like product by any suitable means, such as by spraying, or if the sheet is made by a wet-laying process, by incorporating the salts into the water used to suspend the fibers prior to sheet formation. Additionally, the salts can be added to the sheet as a neat liquid or a solid. The salts will then absorb moisture from the air and distribute throughout the sheet.

In still another embodiment, a deliquescent salt precursor may be incorporated into the tissue sheet and then converted into a deliquescent salt later. For example, a sheet-like product may be made containing calcium carbonate and magnesium carbonate. The sheet-like product may then be sprayed with hydrochloric acid, which converts the calcium carbonate to calcium chloride and the magnesium carbonate to magnesium chloride, water and carbon dioxide. Any residual hydrochloric acid is thereafter removed by drying of the sheet or any other method known in the art. Further, it should be understood that when incorporating a mixture of salts into a sheet-like product, the salts may be added to the sheet sequentially or may be first combined and then added to the sheet.

The salts containing at least one deliquescent salt may be incorporated into any suitable sheet-like material where it is desirable to raise the equilibrium moisture content. As described above, the sheet-like material may comprise a dry product or may comprise a pre-moistened product. The product may also comprise a single ply product or may contain multiple plies such as two or three plies.

In one embodiment, the sheet-like product contains a nonwoven fibrous sheet having a dry sheet bulk of 2 cm³ or greater per gram, such as 3 cm³ or greater per gram, such as about 5 cm³ or greater per gram, or even about 10 cm³ or greater per gram. For example, the dry sheet bulk may be from about 5 to about 25 cm³/g, such as from about 10 to about 20 cm³/g. Particularly suitable nonwoven fibrous sheets include cellulosic or paper sheets useful as facial tissues, bath tissues, paper towels, table napkins, wipes and the like. Other suitable nonwoven fibrous sheets include those consisting essentially of synthetic fibers or comprising a blend of synthetic and natural fibers. Suitable natural hydrophilic fibers include those prepared from polylactic acid.

For the tissue sheets of the present invention, both creped and uncreped webs may be used. Uncreped tissue production is disclosed in U.S. Pat. No. 5,772,845, issued on Jun. 30, 1998 to Farrington, Jr. et al., the disclosure of which is herein incorporated by reference to the extent it is non-contradictory herewith. Creped tissue production is disclosed in U.S. Pat. No. 5,637,194, issued on Jun. 10, 1997 to Ampulski et al.; U.S. Pat. No. 4,529,480, issued on Jul. 16, 1985 to Trokhan; U.S. Pat. No. 6,103,063, issued on Aug. 15, 2000 to Oriaran et al.; and, U.S. Pat. No. 4,440,597, issued on Apr. 3, 1984 to Wells et al., the disclosures of all of which are herein incorporated by reference to the extent that they are non-contradictory herewith. Also suitable for application of the above mentioned chemical additives are tissue sheets that are pattern densified or imprinted, such as the webs disclosed in any of the following U.S. Pat. Nos.: 4,514,345, issued on Apr. 30, 1985 to Johnson et al.; 4,528,239, issued on Jul. 9, 1985 to Trokhan; 5,098,522, issued on Mar. 24, 1992; 5,260,171, issued on Nov. 9, 1993 to Smurkoski et al.; 5,275,700, issued on Jan. 4,1994 to Trokhan; 5,328,565, issued on Jul. 12, 1994 to Rasch et al.; 5,334,289, issued on Aug. 2, 1994 to Trokhan et al.; 5,431,786, issued on Jul. 11, 1995 to Rasch et al.; 5,496,624, issued on Mar. 5,1996 to Steltjes, Jr. et al.; 5,500,277, issued on Mar. 19, 1996 to Trokhan et al.; 5,514,523, issued on May 7, 1996 to Trokhan et al.; 5,554,467, issued on Sep. 10, 1996 to Trokhan et al.; 5,566,724, issued on Oct. 22, 1996 to Trokhan et al.; 5,624,790, issued on Apr. 29, 1997 to Trokhan et al.; and, 5,628,876, issued on May 13, 1997 to Ayers et al., the disclosures of all of which are herein incorporated by reference to the extent that they are non-contradictory herewith. Such imprinted tissue webs may have a network of densified regions that have been imprinted against a drum dryer by an imprinting fabric, and regions that are relatively less densified (e.g., “domes” in the tissue sheet) corresponding to deflection conduits in the imprinting fabric, wherein the tissue sheet superposed over the deflection conduits is deflected by an air pressure differential across the deflection conduit to form a lower-density pillow-like region or dome in the tissue sheet.

Various drying operations may be useful in the manufacture of the tissue products of the present invention. Examples of such drying methods include, but are not limited to, drum drying, through drying, steam drying such as superheated steam drying, displacement dewatering, Yankee drying, infrared drying, microwave drying, radio frequency drying in general, and impulse drying, as disclosed in U.S. Pat. No. 5,353,521, issued on Oct. 11, 1994 to Orloff and U.S. Pat. No. 5,598,642, issued on Feb. 4, 1997 to Orloff et al., the disclosures of both which are herein incorporated by reference to the extent that they are non-contradictory herewith. Other drying technologies may be used, such as methods employing differential gas pressure include the use of air presses as disclosed in U.S. Pat. No. 6,096,169, issued on Aug. 1, 2000 to Hermans et al. and U.S. Pat. No. 6,143,135, issued on Nov. 7, 2000 to Hada et al., the disclosures of both which are herein incorporated by reference to the extent they are non-contradictory herewith. Also relevant are the paper machines disclosed in U.S. Pat. No. 5,230,776, issued on Jul. 27, 1993 to I. A. Andersson et al.

The tissue product may contain a variety of fiber types both natural and synthetic. In one embodiment the tissue product comprises hardwood and softwood fibers. The overall ratio of hardwood pulp fibers to softwood pulp fibers within the tissue product, including individual tissue sheets making up the product may vary broadly. The ratio of hardwood pulp fibers to softwood pulp fibers may range from about 9:1 to about 1:9, more specifically from about 9:1 to about 1:4, and most specifically from about 9:1 to about 1:1. In one embodiment of the present invention, the hardwood pulp fibers and softwood pulp fibers may be blended prior to forming the tissue web thereby producing a homogenous distribution of hardwood pulp fibers and softwood pulp fibers in the z-direction of the tissue web. In another embodiment of the present invention, the hardwood pulp fibers and softwood pulp fibers may be layered (stratified fiber furnish) so as to give a heterogeneous distribution of hardwood pulp fibers and softwood pulp fibers in the z-direction of the tissue web. In another embodiment, the hardwood pulp fibers may be located in at least one of the outer layers of the tissue product and/or tissue webs wherein at least one of the inner layers may comprise softwood pulp fibers. In still another embodiment the tissue product contains secondary or recycled fibers optionally containing virgin or synthetic fibers.

In addition, synthetic fibers may also be utilized in the present invention. The discussion herein regarding pulp fibers is understood to include synthetic fibers. Some suitable polymers that may be used to form the synthetic fibers include, but are not limited to: polyolefins, such as, polyethylene, polypropylene, polybutylene, and the like; polyesters, such as polyethylene terephthalate, poly(glycolic acid) (PGA), poly(lactic acid) (PLA), poly(β-malic acid) (PMLA), poly(ε-caprolactone) (PCL), poly(ρ-dioxanone) (PDS), poly(3-hydroxybutyrate) (PHB), and the like; and, polyamides, such as nylon and the like. Synthetic or natural cellulosic polymers, including but not limited to: cellulosic esters; cellulosic ethers; cellulosic nitrates; cellulosic acetates; cellulosic acetate butyrates; ethyl cellulose; regenerated celluloses, such as viscose, rayon, and the like; cotton; flax; hemp; and mixtures thereof may be used in the present invention. The synthetic fibers may be located in one or all of the layers and sheets comprising the tissue product.

The bone dry basis weight of tissue products treated in accordance with the present invention can also vary depending upon the ultimate use for the product. In general, the bone dry basis weight can range from about 6 gsm to 200 gsm and greater. For example, in one embodiment, the tissue product can have a bone dry basis weight of from about 6 gsm to about 80 gsm.

In addition to dry wiping products, such as tissue products, the salts may also be incorporated into pre-moistened products, such as wet wipes. Such wiping products are broadly known in the art. In one particular embodiment, the pre-moistened wipe is a rolled, moist bath tissue. In other embodiments, however, the pre-moistened wipe may comprise an antimicrobial wiper. For pre-moistened products, the equilibrium moisture content may be increased according to the present invention to greater than 50% by weight of the dry fiber, such as greater than about 100% by weight of the dry fiber. When forming a pre-moistened product, the deliquescent and non-deliquescent salts may be a component of the wetting fluid.

Materials suitable for forming pre-moistened wipes include meltblown, coform, airlaid, bonded carded web materials, hydroentangled materials and the like. The base web may comprise synthetic fibers, natural fibers, or combinations thereof. The base web may have a basis weight of from about 25 gsm to about 120 gsm, such as from about 30 gsm to about 90 gsm. In one particular embodiment, the base sheet comprises a coform web containing polymeric microfibers and cellulosic fibers. Such coform base sheets are manufactured generally as described in U.S. Pat. No. 4,100,324 to Anderson, which is incorporated herein by reference.

Such coform base sheets may, for example, comprise a gas-formed matrix of thermoplastic polymeric meltblown microfibers, such as, for example, polypropylene microfibers, and cellulosic fibers, such as, for example, wood pulp fibers. The relative percentages of the polymeric microfibers and cellulosic fibers in the coform base sheet can vary over a wide range depending upon the desired characteristics of the wet wipes. For example, the coform base sheet may comprise from about 20% to about 100 weight percent, such as from about 20% to about 60 weight percent polymeric microfibers. In one particular embodiment, the polymeric microfibers may comprise from about 30 weight percent to about 40 weight percent.

The pre-moistened wipes are saturated with various liquids as known in the art. In general, the solution incorporated into the pre-moistened wipe depends upon the use of the wipe. Such wet wipes, for example, are used as baby wipes, hand wipes, household cleaning wipes, pre-moistened water dispersible bath tissue, industrial wipes, and the like.

Solutions incorporated into wet wipes have usually included a number of ingredients intended to enhance or impart particular properties to the wipe. These properties have related to, for example, cleaning efficacy, fragrance, medication, reduced irritation, skin health, aesthetics of the product and the like. For baby wipes in particular, a solution providing a gentle soothing feeling without excessive irritation or foam while maintaining cleaning and antimicrobial efficacy is highly desirable for product performance. Suitable ingredients used to provide such properties have included water, emollients, surfactants, preservatives, chelating agents, pH buffers or combinations thereof. The solutions have also contained lotions and/or medicaments.

Various additional chemical additives and ingredients may be incorporated into the sheet-like products without interfering with the deliquescent salts to impart additional benefits. The following materials are included as examples of additional chemicals that may be applied to the sheet-like product.

Friction Reducing Compound

As used herein, a “friction reduction compound” is a material capable of reducing the coefficient of friction (COF) of a non-woven or cellulosic sheet when the non-woven or cellulosic sheet is wetted with water. Particularly useful friction reduction compounds include, without limitation, high molecular weight polyethylene oxide, derivatized polyethylene oxide, cationic acrylamide copolymers having a pendant ethylene oxide moiety, and mixtures thereof.

The amount of the friction reduction compound in the sheets of the products of this invention can be any amount that provides a decrease in the coefficient of friction of the sheet. More specifically, the amount can be about 0.001 weight percent or greater based on the weight of dry fiber, more specifically from about 0.005 to about 10 weight percent, more specifically from about 0.01 to about 5 weight percent and still more specifically from about 0.01 to about 1 weight percent. Typically, amounts greater than about 10 weight percent have a minimal impact on reducing the coefficient of friction.

In one embodiment, the friction reduction compound is a high molecular weight polyethylene oxide. Polyethylene oxides useful for purposes of this invention have the following general formula:
R¹O—(CH₂CH₂O)ₙ R²
wherein R¹ and R² are hydrogen or organo-functional groups. R¹ and R² can be the same or different. These compounds have a weight average molecular weight of about 20,000 or greater, more specifically about 50,000 or greater. In one embodiment, the high molecular polyethylene oxide can have a molecular weight of from about 400,000 to about 2,000,000. As used herein, the molecular weight can be determined by conventional rheological measurements well known in the polymer art.

High molecular weight polyethylene oxides are available from various commercial sources. Examples of polyethylene oxide resins that can be used in the present invention are commercially available from the Union Carbide Corporation and are sold under the trade designations POLYOX N-205, POLYOX-N-750, POLYOX WSR N-10 and POLYOX WSR N-80. The above four products are believed to have weight average molecular weights of from about 100,000 to about 600,000 (g-mol). Polyethylene oxide resins may optionally contain various additives such as plasticizers, processing aids, rheology modifiers, antioxidants, UV light stabilizers, pigments, colorants, slip additives, antiblock agents, etc that may be incorporated in their manufacture.

When treating a sheet with a high molecular weight polyethylene oxide in accordance with the present invention, the high molecular weight polyethylene oxide, for most applications, is applied topically. In general, any suitable topical application process can be used to apply the composition. For example, in one embodiment, the polyethylene oxide can be combined with a solvent such as an alcohol or with water to form a solution and applied to the sheet. When applied as a solution, the composition can be sprayed or printed onto the sheet.

In another embodiment the friction reduction compound can be a derivatized polyethylene oxide, particularly a derivatized high molecular weight polyethylene oxide. For example, polyethylene oxides as described above can be derivatized and used in this embodiment.

A derivatized polyethylene oxide may be formed by reacting a polyethylene oxide with one or more monomers to provide a functional group on the polyethylene oxide polymer. The derivative groups can be placed in the backbone of the polyethylene oxide or can be pendent groups. The derivative groups can be present in the polymer in an amount from about 0.5 percent to about 25 percent by weight, such as from about 0.5% to about 10% by weight.

In one embodiment, a derivatized polyethylene oxide for use in the present invention can be formed by grafting monomers onto the polyethylene oxide. The grafting is accomplished by mixing polyethylene oxide with one or more monomers and an initiator and applying heat. Such treated polyethylene oxide compositions are disclosed in U.S. Pat. No. 6,172,177 issued to Wang et al, which is incorporated herein by reference.

In this embodiment, a variety of polar vinyl monomers may be useful in the practice of the present invention. The term “monomer” as used herein includes monomers, oligomers, polymers, mixtures of monomers, oligomers, and/or polymers, and any other reactive chemical species which is capable of covalent bonding with polyethylene oxide. Ethylenically unsaturated polar vinyl monomers that may be used to derivatize a polyethylene oxide can include as a functional group hydroxyl, carboxyl, amino, carbonyl, halo, thiol, sulfonic, sulfonate, amine, amide, aldehyde, epoxy, silanol, azetidinium groups and the like.

In one embodiment, the unsaturated monomers include acrylates and methacrylates. Such monomers include 2-hydroxyethyl methacrylate (referred to as HEMA) and poly(ethylene glycol) methacrylate. For example, a poly(ethylene glycol) alkyl ether methacrylate can be used, such as poly(ethylene glycol) ethyl ether methacrylate or poly(ethylene glycol) methyl ether methacrylate.

When forming a derivatized polyethylene oxide in this embodiment, an initiator may be useful in forming the polymer. The initiator can generate free radicals when subjected to energy, such as the application of heat. Compounds containing an O—O, S—S, or N═N bond may be used as thermal initiators.

Compounds containing O—O bonds; i.e., peroxides, are commonly used as initiators for graft polymerization. Such commonly used peroxide initiators include: alkyl, dialkyl, diaryl and arylalkyl peroxides such as cumyl peroxide, t-butyl peroxide, di-t-butyl peroxide, dicumyl peroxide, cumyl butyl peroxide, 1,1-di-t-butyl peroxy-3,5,5-trimethylcyclohexane, 2,5-dimethyl-2,5-di(t-butylperoxy)hexane, 2,5-dimethyl-2,5-bis(t-butylperoxy)hexyne-3 and bis(a-t-butyl peroxyisopropylbenzene); acyl peroxides such as acetyl peroxides and benzoyl peroxides; hydroperoxides such as cumyl hydroperoxide, t-butyl hydroperoxide, p-methane hydroperoxide, pinane hydroperoxide and cumene hydroperoxide; peresters or peroxyesters such as t-butyl peroxypivalate, t-butyl peroctoate, t-butyl perbenzoate, 2,5-dimethylhexyl-2,5-di(perbenzoate) and t-butyl di(perphthalate); alkylsulfonyl peroxides; dialkyl peroxymonocarbonates; dialkyl peroxydicarbonates; diperoxyketals; ketone peroxides such as cyclohexanone peroxide and methyl ethyl ketone peroxide. Additionally, azo compounds such as 2,2′-azobisisobutyronitrile (abbreviated as “AIBN”), 2,2′-azobis(2,4-dimethylpentanenitrile) and 1,1′-azobis(cyclohexanecarbonitrile) may be used as the initiator.

In one particular embodiment, the polyethylene oxide polymer is grafted with an amount of an organic moiety that includes a group that reacts with water to form a silanol group. For example, one such functional group that can react with water to form a silanol group is a trialkoxy silane functional group. The trialkoxy silane functional group can have the following structure: 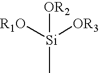
wherein R₁, R₂ and R₃ are the same or different alkyl groups, each independently having 1 to 6 carbon atoms.

In forming derivatized polyethylene oxides that contain a silanol group, the polyethylene oxide can be reacted with a monomer containing, for instance, a trialkoxy silane functional group as illustrated above. For example, in one embodiment, the monomer is an acrylate or methacrylate, such as methacryloxypropyl trimethoxy silane. Methacryloxypropyl propyl trimethoxy silane is commercially available from Dow Corning out of Midland, Mich. under the trade designation Z-6030 Silane.

Other suitable monomers containing a trialkoxy silane functional group include, but are not limited to, methacryloxyethyl trimethoxy silane, methacryloxypropyl triethoxy silane, methacryloxypropyl tripropoxy silane, acryloxypropylmethyl dimethoxy silane, 3-acryloxypropyl trimethoxy silane, 3-methacryloxypropylmethyl diethoxy silane, 3-methacryloxypropylmethyl dimethoxy silane, and 3-methacryloxypropyl tris(methoxyethoxy) silane. However, it is contemplated that a wide range of vinyl and acrylic monomers having trialkoxy silane functional groups or a moiety that reacts easily with water to form a silanol group, such as a chlorosilane or an acetoxysilane, provide the desired effects to PEO and are effective monomers for grafting in accordance with the copolymers of the present invention.

Charge Control Agents

Charge promoters and control agents are commonly used in the papermaking process to control the zeta potential of the papermaking furnish in the wet end of the process. These species may be anionic or cationic, most usually cationic, and may be either naturally occurring materials such as alum or low molecular weight high charge density synthetic polymers typically of molecular weight of about 500,000 or less. Drainage and retention aids may also be added to the furnish to improve formation, drainage and fines retention. Included within the retention and drainage aids are microparticle systems containing high surface area, high anionic charge density materials.

Strength Agents

Wet and dry strength agents may also be applied to the tissue sheet. As used herein, “wet strength agents” refer to materials used to immobilize the bonds between fibers in the wet state. Typically, the means by which fibers are held together in paper and tissue products involve hydrogen bonds and sometimes combinations of hydrogen bonds and covalent and/or ionic bonds. In the present invention, it may be useful to provide a material that will allow bonding of fibers in such a way as to immobilize the fiber-to-fiber bond points and make them resistant to disruption in the wet state. In this instance, the wet state usually will mean when the product is largely saturated with water or other aqueous solutions, but could also mean significant saturation with body fluids such as urine, blood, mucus, menses, runny bowel movement, lymph, and other body exudates.

Any material that when added to a tissue sheet or sheet results in providing the tissue sheet with a mean wet geometric tensile strength:dry geometric tensile strength ratio in excess of about 0.1 will, for purposes of the present invention, be termed a wet strength agent. Typically these materials are termed either as permanent wet strength agents or as “temporary” wet strength agents. For the purposes of differentiating permanent wet strength agents from temporary wet strength agents, the permanent wet strength agents will be defined as those resins which, when incorporated into paper or tissue products, will provide a paper or tissue product that retains more than 50% of its original wet strength after exposure to water for a period of at least five minutes. Temporary wet strength agents are those which show about 50% or less than, of their original wet strength after being saturated with water for five minutes. Both classes of wet strength agents find application in the present invention. The amount of wet strength agent added to the pulp fibers may be at least about 0.1 dry weight percent, more specifically about 0.2 dry weight percent or greater, and still more specifically from about 0.1 to about 3 dry weight percent, based on the dry weight of the fibers.

Permanent wet strength agents will typically provide a more or less long-term wet resilience to the structure of a tissue sheet. In contrast, the temporary wet strength agents will typically provide tissue sheet structures that had low density and high resilience, but would not provide a structure that had long-term resistance to exposure to water or body fluids.

Wet and Temporary Wet Strength Agents

The temporary wet strength agents may be cationic, nonionic or anionic. Such compounds include PAREZ™ 631 NC and PAREZ® 725 temporary wet strength resins that are cationic glyoxylated polyacrylamide available from Cytec Industries (West Paterson, N.J.). This and similar resins are described in U.S. Pat. No. 3,556,932, issued on Jan. 19, 1971 to Coscia et al. and U.S. Pat. No. 3,556,933, issued on Jan. 19, 1971 to Williams et al. Hercobond1366, manufactured by Hercules, Inc., located at Wilmington, Del., is another commercially available cationic glyoxylated polyacrylamide that may be used in accordance with the present invention. Additional examples of temporary wet strength agents include dialdehyde starches such as Cobond® 1000 from National Starch and Chemical Company and other aldehyde containing polymers such as those described in U.S. Pat. No. 6,224,714 issued on May 1, 2001 to Schroeder et al.; U.S. Pat. No. 6,274,667 issued on Aug. 14, 2001 to Shannon et al.; U.S. Pat. No. 6,287,418 issued on Sep. 11, 2001 to Schroeder et al.; and, U.S. Pat. No. 6,365,667 issued on Apr. 2, 2002 to Shannon et al., the disclosures of which are herein incorporated by reference to the extend they are non-contradictory herewith.

Permanent wet strength agents comprising cationic oligomeric or polymeric resins can be used in the present invention. Polyamide-polyamine-epichlorohydrin type resins such as KYMENE 557H sold by Hercules, Inc., located at Wilmington, Del., are the most widely used permanent wet-strength agents and are suitable for use in the present invention. Such materials have been described in the following U.S. Pat. Nos.: 3,700,623 issued on Oct. 24, 1972 to Keim; 3,772,076 issued on Nov. 13, 1973 to Keim; 3,855,158 issued on Dec. 17, 1974 to Petrovich et al.; 3,899,388 issued on Aug. 12, 1975 to Petrovich et al.; 4,129,528 issued on Dec. 12, 1978 to Petrovich et al.; 4,147,586 issued on Apr. 3, 1979 to Petrovich et al.; and, 4,222,921 issued on Sep. 16, 1980 to van Eenam. Other cationic resins include polyethylenimine resins and aminoplast resins obtained by reaction of formaldehyde with melamine or urea. It is often advantageous to use both permanent and temporary wet strength resins in the manufacture of tissue products with such use being recognized as falling within the scope of the present invention.

Dry Strength Agents

Dry strength agents may also be applied to the tissue sheet without affecting the performance of the present invention. Such materials used as dry strength agents are well known in the art and include but are not limited to modified starches and other polysaccharides such as cationic, amphoteric, and anionic starches and guar and locust bean gums, modified polyacrylamides, carboxymethylcellulose, sugars, polyvinyl alcohol, chitosan, and the like. Such dry strength agents are typically added to a fiber slurry prior to tissue sheet formation or as part of the creping package.

Additional Softening Agents

At times it may be advantageous to add additional debonders or softening chemistries to a tissue sheet. Examples of such debonders and softening chemistries are broadly taught in the art. Exemplary compounds include the simple quaternary ammonium salts having the general formula (R^{1′})_{4-b} N⁺(R^{1″})_{b} X⁻ wherein R^{1,} is a C₁₋₆ alkyl group, R^{1″} is a C₁₄-C₂₂ alkyl group, b is an integer from 1 to 3 and X− is any suitable counterion. Other similar compounds include the monoester, diester, monoamide and diamide derivatives of the simple quaternary ammonium salts. A number of variations on these quaternary ammonium compounds are known and should be considered to fall within the scope of the present invention. Additional softening compositions include cationic oleyl imidazoline materials such as methyl-1-oleyl amidoethyl-2-oleyl imidazolinium methylsulfate commercially available as Mackernium DC-183 from Mcintyre Ltd., located in University Park, Ill. and Prosoft TQ-1003 available from Hercules, Inc. Other exemplary softening agents which may be used to further enhance the softness of the tissue webs of the present invention include polysiloxanes. Such polysiloxanes being broadly described in the art for enhancing the softness of tissue webs.

Miscellaneous Agents

In general, the present invention may be used in conjunction with any known materials and chemicals that are not antagonistic to its intended use. Examples of such materials and chemicals include, but are not limited to, odor control agents, such as odor absorbents, activated carbon fibers and particles, baby powder, chelating agents, zeolites, perfumes or other odor-masking agents, cyclodextrin compounds, oxidizers, and the like. Superabsorbent particles, synthetic fibers, or films may also be employed. Additional options include cationic dies, optical brighteners, absorbency aids and the like. A wide variety of other materials and chemicals known in the art of papermaking and tissue production may be included in the tissue sheets of the present invention including lotions and other materials providing skin health benefits including but not limited to such things as aloe extract and tocopherols such as Vitamin E and the like. The addition of various additives may require use of specific deliquescent salts to avoid any negative interactions such as formation of insoluble precipitates caused by interaction of the deliquescent salt and the additive.

The application point for such materials and chemicals is not particularly relevant to the present invention and such materials and chemicals may be applied at any point in the tissue manufacturing process. This includes pre-treatment of pulp, co-application in the wet end of the process, post treatment after drying but on the tissue machine and topical post treatment.

The present invention may be better understood with reference to the following examples.

### EXAMPLE 1

The following example was conducted in order to demonstrate that by mixing at least one deliquescent salt with other salts, the deliquescence point may be controlled and lowered in comparison to the deliquescence point of each of the individual salts.

In this example, salt mixtures were made from calcium chloride, sodium chloride, magnesium chloride, and potassium acetate. Specifically, the following solutions were prepared:

CaCl₂—prepared from the hexahydrate. Concentration of CaCl₂ (anhydrous) was 34.7%. The deliquescence point of CaCl₂ is approximately 30% relative humidity.

MgCl₂—prepared from anhydrous salt. Concentration of MgCl₂ (anhydrous) was 32.2%. The deliquescence point of MgCl2 is approximately 32.8% relative humidity.

NaCl—prepared from the anhydrous salt. Concentration of NaCl (anhydrous) was 23.8%. The deliquescence point of NaCl is approximately 75.3% relative humidity.

KC₂H₃O₂—The deliquescence point of KC₂H₃O₂ is approximately 22.5% relative humidity.

The solutions were blended in the following ratios and allowed to sit in a fume hood at an ambient humidity of 15±3% for 24 hours at a temperature of 70±3° F. Table 1 summarizes the results.

TABLE 1

| | | | | | Observations - |
|---|---|---|---|---|---|
| | | | | | 24 hours @ 15% |
| Sample ID | CaCl₂ | NaCl | MgCl₂ | KC₂H₃O₂ | RH |
| A (Control) | 2.00 | x | x | X | Glassy Solid |
| B (Control) | x | 2.00 | x | X | Powdery solid |
| | | | | | started forming at |
| | | | | | 1 hour. |
| C | 1.80 | 0.20 | x | X | Glassy/opaque |
| | | | | | solid |
| D | 1.58 | 0.55 | x | X | Quite liquid - |
| | | | | | small amount of |
| | | | | | solid |
| E | 1.00 | 1.14 | x | x | Wet solid. |
| F | 1.81 | x | 0.21 | x | Dry somewhat |
| | | | | | glassy solid. |
| G | 1.49 | x | 0.53 | x | Wet solid. |
| H | 1.06 | x | 0.99 | x | Glassy wet solid, |
| | | | | | small amount of |
| | | | | | liquid. |
| J | 0.49 | x | 1.62 | x | Highly liquid, |
| | | | | | small amount of |
| | | | | | glassy solid. |
| K (Control) | x | x | 1.00 | x | Wet solid. |
| L | x | 0.27 | 1.82 | x | Highly liquid, |
| | | | | | some cloudiness. |
| M | x | 0.60 | 1.49 | X | Highly liquid, |
| | | | | | some cloudiness. |
| N | 0.81 | 0.16 | 0.78 | x | Solid, some |
| | | | | | liquid. |
| P | 0.81 | 0.12 | 0.96 | x | Solid, some |
| | | | | | liquid. |
| Q (Control) | x | x | x | 1.35 | Glassy solid, |
| | | | | | small amount of |
| | | | | | liquid. |
| R | x | 0.60 | x | 1.50 | Wet solid. |
| S | x | x | 0.53 | 1.53 | Wet solid. |
| T | x | 0.16 | x | 1.88 | Wet solid. |

As shown above, many of the mixtures remained in a wet state even at a relative humidity of 15%.

### EXAMPLE 2

Based on the results in Example No. 1, further tests were completed using the same salts and changing the weight ratios.

Specifically, various mixtures containing at least one deliquescent salt were prepared and were left standing at 12%±2% relative humidity for at least 21 days at 70±2° F. In this example, the percent solids of each mixture was calculated before and after being removed from the fume hood. Specifically, the samples were measured for weight loss.

For purposes of comparison, two controls were also prepared that contained a single salt. In particular, Control No. 1 contained only calcium chloride, while Control No. 2 only contained sodium chloride.

Sample No. 7 below was prepared by mixing 11.46 grams of magnesium chloride solution and 5.87 grams of sodium chloride solution and allowing for a precipitate to form and settle. The supernatant liquid was withdrawn and used in this example. The supernatant represents the equilibrium ratio of magnesium chloride and sodium chloride in solution at 70° F.

The following results were obtained:

TABLE 2

| | | | | | Water | % Solids | |
|---|---|---|---|---|---|---|---|
| Sample | | | | % | Loss | After | |
| # | MgCl₂ | NaCl | CaCl₂ | Solids | (g) | Drying | Observations |
| 1 | 1.75 | 0.00 | 0.17 | 45.6% | 0.16 | 49.8% | Glassy liquid + |
| | | | | | | | solid − flows |
| 2 | 1.51 | 0.00 | 0.38 | 44.3% | 0.28 | 52.0% | Glassy liquid + |
| | | | | | | | solid − flows |
| 3 | 1.88 | 0.00 | 0.09 | 46.2% | 0.19 | 51.1% | Glassy liquid + |
| | | | | | | | solid − flows |
| 4 | 1.89 | 0.09 | 0.00 | 46.2% | 0.20 | 51.3% | Flows, small |
| | | | | | | | amount of |
| | | | | | | | solid |
| 5 | 1.77 | 0.22 | 0.00 | 45.4% | 0.25 | 51.9% | Flows, small |
| | | | | | | | amount of |
| | | | | | | | solid |
| 6 | 1.37 | 0.62 | 0.00 | 43.0% | 0.54 | 59.0% | No flow, wet |
| | | | | | | | solid |
| 7 | 1.96 | | 0.00 | 32.6% | 0.57 | 46.0% | Very liquid, |
| | | | | | | | flows well, |
| | | | | | | | no solid. |
| Control | 0.00 | 0.00 | 2.00 | 34.7% | 1.00 | 69.4% | Glassy solid, |
| 1 | | | | | | | no flow |
| Control | 0.00 | 2.00 | 0.00 | 23.4% | 1.51 | 95.5% | Powdery |
| 2 | | | | | | | solid |

### EXAMPLE 3

The following example was conducted to test the ability of lithium bromide to deliquesce at low relative humidity conditions.

In this example, 1.0 grams of lithium bromide (99%, anhydrous obtained from Aldrich Chemical) was placed in an aluminum weighing boat and allowed to stand at 14% relative humidity and 72° F. for 24 hours. After the 24 hour period, it was observed that the lithium bromide absorbed 0.91 grams of water and formed a free flowing clear solution.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A tissue product comprising:
a tissue web containing cellulosic fibers, the tissue web having a dry sheet bulk of at least 2 cm³/g; and
a moisture retaining material comprising a mixture of deliquescent salts, a mixture of deliquescent and non-deliquescent salts, or lithium bromide in association with the tissue web, the moisture retaining material being present in an amount sufficient such that the tissue web has an equilibrium moisture content of greater than about 10% at a relative humidity of less than about 30% at 72° F.

2. A tissue product as defined in claim 1, wherein the moisture retaining material comprises a mixture of at least two salts selected from the group consisting of calcium chloride, magnesium chloride, potassium acetate, lithium bromide, lithium chloride, potassium carbonate, magnesium nitrate, sodium bromide, potassium iodide, sodium chloride, potassium chloride, potassium sulfate, an aluminate, sodium acetate, and ammonium acetate.

3. A tissue product as defined in claim 1, wherein the product comprises a facial tissue or a bath tissue.

4. A tissue product as defined in claim 1, wherein the tissue web has an equilibrium moisture content of from about 10% to about 30% at relative humidities between 5% and 30% at 72° F.

5. A wet wipe comprising:
a base web containing a wiping solution; and
a moisture retaining material comprising a mixture of deliquescent salts, a mixture of deliquescent and non-deliquescent salts, or lithium bromide in association with the base web, the moisture retaining material having an equilibrium moisture content of greater than about 50% at relative humidities between 15% and 30% at 72° F.

6. A wet wipe as defined in claim 5, wherein the base web has an equilibrium moisture content of greater than about 100% at relative humidities between 15% and 30% at 72° F.

7. A wet wipe as defined in claim 5, wherein the base web has an equilibrium moisture content of greater than about 150% at relative humidities between 15% and 30% at 72° F.

8. A wet wipe as defined in claim 5, wherein the moisture retaining material comprises a mixture of at least two salts selected from the group consisting of calcium chloride, magnesium chloride, potassium acetate, lithium bromide, lithium chloride, potassium carbonate, magnesium nitrate, sodium bromide, potassium iodide, sodium chloride, potassium chloride, potassium sulfate, an aluminate, sodium acetate, and ammonium acetate.

9. A fibrous product having an equilibrium moisture content of greater than about 10% at a relative humidity of less than about 30%.

10. The fibrous product of claim 9 having an equilibrium moisture content of greater than 10% at a relative humidity of less than about 20%.

11. The fibrous product of claim 9 comprising:
a base web containing cellulosic or non-cellulosic fibers; and
a mixture of deliquescent salts, a mixture of deliquescent and non-deliquescent salts or lithium bromide wherein the salt or salt mixture has a deliquescence point of less than 30%.

12. The fibrous product of claim 11, wherein the base web contains a mixture of salts and the deliquescence point of the salt mixture is lower than the deliquescent point of the individual salts in the mixture.

13. A fibrous product as defined in claim 9, wherein the base web comprises a tissue web consisting essentially of pulp fibers, the tissue web having a dry sheet bulk of at least about 2 cm³/g.

14. The fibrous product of claim 13, wherein the fibrous product has an equilibrium moisture content of from about 10% to about 30% by weight of dry fibers.

15. A fibrous product as defined in claim 11, wherein the base web contains a mixture of salts that comprise at least two salts selected from the group consisting of calcium chloride, magnesium chloride, potassium acetate, lithium bromide, lithium chloride, potassium carbonate, magnesium nitrate, sodium bromide, potassium iodide, sodium chloride, potassium chloride, potassium sulfate, an aluminate, sodium acetate, and ammonium acetate.

16. A fibrous product as defined in claim 11, wherein the base web contains a mixture of salts comprising calcium chloride, magnesium chloride, or mixtures thereof.

17. A fibrous product as defined in claim 9, wherein the fibrous product comprises a deliquescent salt comprising lithium bromide.

18. A fibrous product as defined in claim 11, wherein the base web contains a mixture of salts that have a deliquescence point of less than about 25%.

19. A fibrous product as defined in claim 9, wherein the base web comprises a tissue web and wherein the base web has an equilibrium moisture content of from about 10% to about 30% at a relative humidity between 5% and 30% at 72° F.

20. A fibrous product as defined in claim 9, wherein the product comprises a wet wipe and the base web has an equilibrium moisture content of from about 30% to about 200% at a relative humidity between 5% and 30% at 72° F.

21. A fibrous product as defined in claim 11, wherein the base web contains a mixture of salts that comprises a mixture of at least three different salts.

22. A fibrous product as defined in claim 11, wherein the base web contains a mixture of salts and the mixture of salts comprises a mixture of at least two metal chloride salts.

23. A fibrous product as defined in claim 9, wherein the base web further contains a friction reducing agent.

24. A method for making a fibrous product having an equilibrium moisture content of greater than about 10% at a relative humidity of less than about 30%, said method comprising:
a) forming a fibrous web;
b) adding a mixture of deliquescent salts or a mixture of deliquescent and non-deliquescent salts to the fibrous web wherein the deliquescence point of the salt mixture is selected such that the deliquescence point of the mixture is less than 30%.

25. The method of claim 24, wherein the deliquescence point of the salt mixture is lower than the deliquescence point of the individual salts in the mixture.

26. The method of claim 24, wherein the deliquescence point of the salt mixture is less than about 20%.

27. The method of claim 24, wherein the deliquescent salt mixture comprises at least one non-deliquescent salt.

28. The method of claim 24, wherein the salt mixture comprises at least two salts selected from the group consisting of calcium chloride, magnesium chloride, potassium acetate, lithium bromide, lithium chloride, potassium carbonate, magnesium nitrate, sodium bromide, potassium iodide, sodium chloride, potassium chloride, potassium sulfate, an aluminate, sodium acetate, and ammonium acetate.

29. The method of claim 24, wherein the salt mixture comprises calcium chloride, magnesium chloride, or mixtures thereof.
